(19) United States
(12) Patent Application Publication
(10) Pub. No.: US 2025/0222005 A1
(43) Pub. Date: Jul. 10, 2025

(54) METHYLENE BLUE CONTAINING COMPOUNDS FOR THE TREATMENT OF METHAEMOGLOBINAEMIA

(71) Applicant: Wis Ta Laboratories Ltd., Singapore (SG)

(72) Inventors: Charles Robert Harrington, Aberdeenshire (GB); Mohammad Arastoo, Aberdeenshire (GB); Michael Philip Mazanetz, Glasgow (GB); Helen Shiells, Aberdeen, Aberdeenshire (GB); John Mervyn David Storey, Old Aberdeen, Aberdeenshire (GB); Claude Michel Wischik, Aberdeen, Aberdeenshire (GB)

(73) Assignee: Wis Ta Laboratories Ltd., Singapore (SG)

(21) Appl. No.: 18/849,744

(22) PCT Filed: Mar. 16, 2023
(86) PCT No.: PCT/EP2023/056759

§ 371 Date: Sep. 23, 2024

(30) Foreign Application Priority Data
Mar. 24, 2022 (GB) 2204185.9

(51) Int. Cl. A61K 31/5415; A61K 9/00; A61P 7/00

(52) U.S. Cl. CPC A61K 31/5415; A61K 9/0053; A61P 7/00

(57) ABSTRACT

The invention provides novel methods of treating methaemoglobinaemia orally in a subject, which methods comprise orally administering to said subject a methylthioninium (“MT”) containing salt of the following formula: wherein each of HnA and HnB (where present) are protic acids which may be the same or different, and wherein p=1 or 2; q=0 or 1; n=1 or 2; (p+q)×n=2, or a hydrate or solvate thereof. The administration should provide a total daily oral dose of 4 mg to 60 mg of MT to the subject per day.

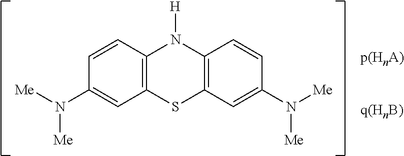

### CROSS-REFERENCE TO RELATED APPLICATIONS

This patent application claims the benefit of priority of GB 2204185.9 filed on 24 Mar. 2022 and which is herein incorporated in its entirety.

### TECHNICAL FIELD

The present invention relates generally to methods and materials for use alleviating methaemoglobinaemia or treatment of methaemoglobinaemia in a subject.

### BACKGROUND ART

Methaemoglobinaemia is a rare disorder associated with oxidization of divalent ferro-iron of haemoglobin to ferri-iron of methaemoglobin (see “Iolascon, Achille, et al. “Recommendations for diagnosis and treatment of methemoglobinemia.” American Journal of Hematology (2021) 96: 1666-1678.”).

The presence of iron in the ferric [Fe³⁺] state results in allosteric changes that permit the binding of oxygen irreversibly. The corresponding ferro-globins in the tetramer shifts the oxygen dissociation curve of Hb to the left. This shift leads to increased affinity of the ferrous iron for oxygen and thus impaired oxygen release to the tissue, resulting in hypoxia with the so called “functional anaemia” without Hb decrease.

Methaemoglobinaemia can result from either inherited or acquired processes. Acquired forms are the most common, mainly due to the exposure to substances that cause oxidation of the Hb both directly or indirectly. Inherited forms are due either to autosomal recessive variants in the CYB5R3 gene or to autosomal dominant variants in the globin genes, collectively known as haemoglobinaemia disease.

Intravenous methylthionium chloride (MTC, methylene blue) is a standard treatment for methaemoglobinamia (see “Skold, A., et al. “Methemoglobinemia.” Southern Medical Journal 2011; 104:757-761.”). The methylthionine (MT) moiety can exist in the oxidised MT⁺ form (in MTC) and in the reduced or “leuco” form hydromethylthionine (LMT) form (see “Harrington, C. R., et al. “Cellular models of aggregation-dependent template-directed proteolysis to characterize tau aggregation inhibitors for treatment of Alzheimer disease.” Journal of Biological Chemistry 2015; 290:10862-10875.”). MT⁺ needs to first be converted to LMT to permit absorption, distribution and uptake into cells (see “Baddeley, T. C., et al. “Complex disposition of methylthioninium redox forms determines efficacy in tau aggregation inhibitor therapy for Alzheimer's disease.” Journal of Pharmacology and Experimental Therarapeutics. 2015; 352:110-118,” and “Merker, M. P., et al. “Pulmonary endothelial thiazine uptake: separation of cell surface reduction from intracellular reoxidation.” American Journal of Physiology: Lung Cellular and Molecular Physiology. 1997; 272:L673-L680.”) LMT is the active species at the heme site of action where it facilitates the transfer of an electron from LMT to Fe³⁺, reducing it to Fe²⁺ and oxidising LMT to MT⁺ in the process. The continuing regeneration of LMT via ongoing red cell glycolysis permits the restoration of normal oxygen-carrying capacity (see “Blank, O., et al. “Interactions of the antimalarial drug methylene blue with methemoglobin and heme targets in Plasmodium falciparum: A physico-biochemical study.” Antioxidants and Redox Signaling. 2012; 17:544-554,” and Yubisui, T., et al. “Reduction of methemoglobin through flavin at the physiological concentration by NADPH-flavin reductase of human erythrocytes.” Journal of Biochemistry 1980; 87:1715-1720.)

However the role of MTC in the treatment of methaemoglobinaemia is paradoxical. For example MTC can actually cause acquired methaemoglobinaemia (See Iolascon 2021, supra).

It has been reported that methylene blue-vitamin C—N-acetyl Cysteine (MCN) provided benefits to critically ill COVID-19 patients, with one presumptive mechanism of this agent and dosage being via reduction in methaemoglobin (metHb) (Alamdari, Daryoush Hamidi, et al. “Application of methylene blue-vitamin C—N-acetyl cysteine for treatment of critically ill COVID-19 patients, report of a phase-I clinical trial.” European Journal of Pharmacology 885 (2020): 173494.

WO2021/224146 concerns the use of hydromethylthionine and related salts (referred to as “LMTX” therein) as therapeutics for alleviating hypoxemia in subjects. It is proposed that these salts may therefore be used to alleviate hypoxia and treat pathologies or other causes of hypoxia. The effects disclosed therein are said to be unrelated to any known effects on metHb, and it is noted in WO2021/224146 that LMT at high concentrations (associated with oral doses in the range 150-250 mg/day) can produce a measurable increase in metHb. The results described in WO2021/224146 indicated that an LMTX compound at high dosage over a period of time systematically increased metHb levels (see FIG. 3 of WO2021/224146) while nevertheless alleviating hypoxia.

It can be seen from the foregoing that providing compounds which can be used safely and conveniently to treat methaemoglobinaemia (i.e. reduce levels of metHb in a subject) would provide a useful contribution to the art.

### DISCLOSURE OF THE INVENTION

The present invention concerns the use of “LMTX” compounds, including LMT, delivered orally at an appropriate dosage, to treat methaemoglobinaemia (i.e. reduce levels of methaemoglobin) in a subject. The treatment is irrespective of, and independent of, the presence or absence of hypoxia. This provides a more convenient way to treat methaemoglobinaemia than the use of intravenous MTC.

Oral LMTX have not previously been disclosed for the treatment of methaemoglobinaemia. However it is the insight of the present inventors that the nature of the LMT binding interaction with the Fe³⁺ of ferro-globin is such that oral treatment, at appropriate dose, can provide benefit in terms of conversion to the ferri-globin.

The oral treatment may be applied to either of hereditary or acquired methaemoglobinaemia. The availability of an oral treatment with a benign safety profile for use in these conditions would represent a valuable addition to the treatment options currently available.

Thus in one aspect there is disclosed a method of treating (or alleviating) methaemoglobinaemia in a subject,
which method comprises orally administering to said subject a methylthioninium (MT)-containing compound,
wherein said administration provides a total daily oral dose of 4 mg to 60 mg of MT to the subject per day, optionally split into 2 or more doses,
wherein the MT-containing compound is an LMTX compound of the following formula:

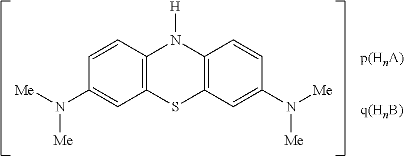

wherein each of HₙA and HₙB (where present) are protic acids which may be the same or different,
and wherein p=1 or 2; q=0 or 1; n=1 or 2; (p+q)×n=2, or a hydrate or solvate thereof.

The total daily MT dose may be between 8 or 10 or 20 or 20.5 or 21 and 50 or 60 mg.

The total daily dose may be about 4, 8, 12, 16, 20, 20.5, 21, 21.5, 22, 22.5, 23, 23.5, 24 mg to around any of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 mg.

The total daily dose may be about 8, 12, 16, 20, 20.5, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg.

An example dosage is 10 to 50 mg.

A further example dosage is 20 or 20.5 to 50 mg.

A further example dosage is 30 to 50 mg.

A further example dosage is 40 to 50 mg.

The total daily dose of the compound may be administered as a split dose twice a day or three times a day.

As explained below, when administering the MT dose split in a larger number of doses/day it may be desired to use a smaller total amount within the recited range, compared to a single daily dosing, or a smaller number of doses per day.

The patient may be an adult human, and the population-based dosages described herein are premised on that basis (typical weight 50 to 70 kg). If desired, corresponding dosages may be utilised for subjects falling outside of this range by using a subject weight factor whereby the subject weight is divided by 60 kg to provide the multiplicative factor for that individual subject.

The subject for treatment may be characterised or selected by certain criteria.

Signs, symptoms, and causes of methemoglobinemia used for selection may include any of those in Table 7 (See Iolascon 2021, supra):

In one embodiment the subject is not hypoxic or suffering from hypoxaemia.

In one embodiment the subject has a blood oxygen saturation level (SpO2) of more than 95% on room air.

For the present invention the subject must be able to breathe and swallow as the treatment is to be administered orally.

The methods of the invention may comprise the step of selecting the subject according to one or more of the above criteria

Thus in some embodiments the subject may be a human who has been diagnosed as having methaemoglobinaemia, or wherein said method comprises making said diagnosis.

The invention may be applied in treating acquired or hereditary forms of methaemoglobinaemia. Examples of diseases of acquired and hereditary forms of methaemoglobinaemia are shown in Table 6. Thus the treatment may be for methaemoglobinaemia resulting from drug exposure (e.g. Phenazopyridine (Pyridium), Sulfamethoxazole, Dapsone, Aniline, Paraquat/monolinuron, Nitrate, Nitroglycerin, Amyl nitrite, Isobutyl nitrite, Sodium nitrite, Benzocaine, lidocaine, Prilocaine, or Chloramine).

In some embodiments the hereditary disease is type I, II, III, or IV methaemoglobinaemia, or HbM disease, or unstable haemoglobin.

In some embodiments the subject has a metHb level of greater than 20%.

Methaemoglobinaemia symptoms may depend on the rapidity of metHb formation. For example patients with lifelong methemoglobinemia may be symptomatic, but patients exposed to drugs and toxins who abruptly develop the same levels of methemoglobinemia may be severely symptomatic.

In some embodiments the subject has a metHb level as described in Table 7. For example the subject may be symptomatic with a metHb level of great than 30%, 50% or 70%.

In some embodiments the subject has acute methaemoglobinaemia.

In some embodiments the subject has chronic methaemoglobinaemia.

In some embodiments the hereditary disease is autosomal recessive congenital methaemoglobinaemia.

WO2007/110627 disclosed certain 3,7-diamino-10H-phenothiazinium salts, effective as drugs or pro-drugs for the treatment of diseases including Alzheimer's disease and other diseases such as Frontotemporal dementia (FTD), as well as viral diseases generally. These compounds are also in the “reduced” or “leuco” form when considered in respect of MTC. These leucomethylthioninium compounds were referred to therein as “LMTX” salts.

WO2012/107706 described other LMTX salts having superior properties to the LMTX salts listed above, including leuco-methylthioninium bis(hydromethanesulfonate) (LMTM) (WHO INN designation: hydromethylthionine):

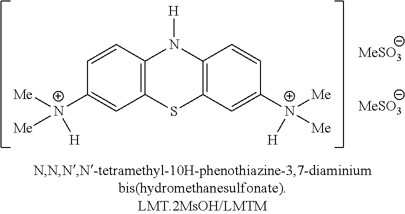

Preferably the LMT compound is an “LMTX” compound of the type described in WO2007/110627 or WO2012/107706.

Thus the compound may be selected from compounds of the following formula, or hydrates or solvates thereof:

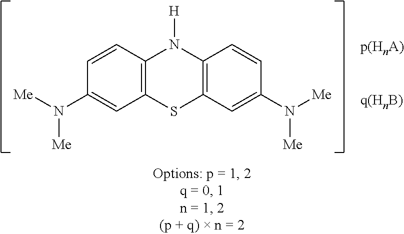

Each of HₙA and HₙB (where present) are protic acids which may be the same or different.

By “protic acid” is meant a proton (H⁺) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

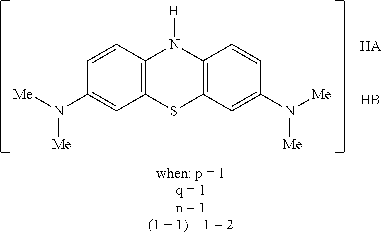

However preferably the salt is not a mixed salt, and has the following formula:

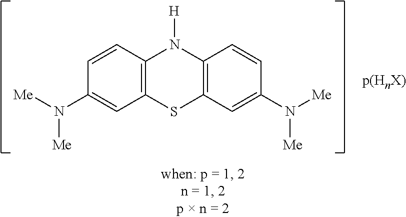

wherein each of HₙX is a protic acid, such as a di-protic acid or mono-protic acid.

In one embodiment the salt has the following formula, where H₂A is a di-protic acid:

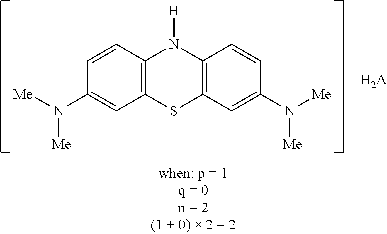

Preferably the salt has the following formula which is a bis monoprotic acid:

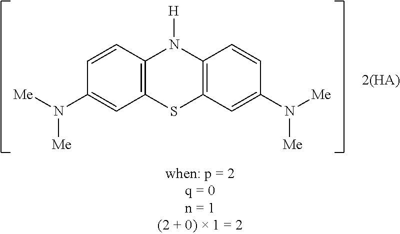

Examples of protic acids which may be present in the LMTX compounds used herein include:
Inorganic acids: hydrohalide acids (e.g., HCl, HBr), nitric acid (HNO₃), sulphuric acid (H₂SO₄)
Organic acids: carbonic acid (H₂CO₃), acetic acid (CH₃COOH), methanesulfonic acid, 1,2-ethanedisulfonic acid, ethansulfonic acid, naphthalenedisulfonic acid, p-toluenesulfonic acid,

Preferred acids are monoprotic acid, and the salt is a bis(monoprotic acid) salt.

A preferred MT compound is LMTM:

| | | | |
|---|---|---|---|
| 1 | 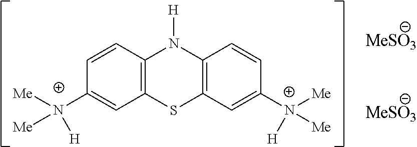 | LMT.2MsOH (LMTM) | 477.6 (1.67) |

### Weight Factors

The anhydrous salt has a molecular weight of around 477.6. Based on a molecular weight of 285.1 for the LMT core, the weight factor for using this MT compound in the invention is 1.67. By “weight factor” is meant the relative weight of the pure MT-containing compound vs. the weight of MT which it contains.

Other weight factors can be calculated for example MT compounds herein, and the corresponding dosage ranges can be calculated therefrom.

Other example LMTX compounds are as follows. Their molecular weight (anhydrous) and weight factor is also shown:

| | | | |
|---|---|---|---|
| 2 | 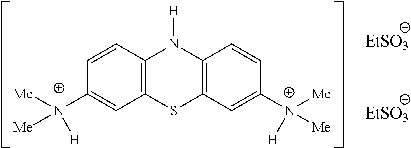 | LMT.2EsOH | 505.7 (1.77) |
| 3 | 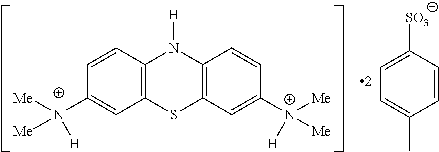 | LMT.2TsOH | 629.9 (2.20) |
| 4 | 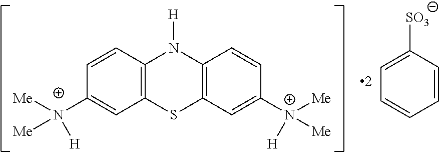 | LMT.2BSA | 601.8 (2.11) |
| 5 | 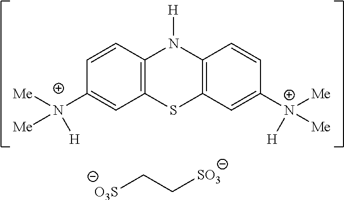 | LMT.EDSA | 475.6 (1.66) |
| 6 | 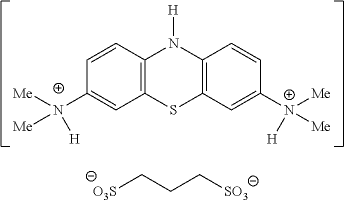 | LMT.PDSA | 489.6 (1.72) |
| 7 | 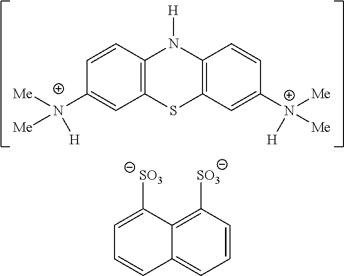 | LMT.NDSA | 573.7 (2.01) |
| 8 | 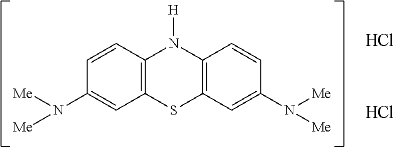 | LMT.2HCl | 358.33 (1.25) |

The dosages described herein with respect to MT thus apply mutatis mutandis for these MT-containing compounds, as adjusted for their molecular weight.

### Accumulation Factors

As will be appreciated by those skilled in the art, for a given daily dosage, more frequent dosing can lead to greater accumulation of a drug.

Therefore in certain embodiments of the claimed invention, the total daily dosed amount of MT compound may be relatively lower, when dosing more frequently (e.g. twice a day [bid] or three times a day [tid]), or higher when dosing once a day [qd].

### Treatment and Prophylaxis

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen. The present inventors have demonstrated that a therapeutically-effective amount of an MT compound in respect of the diseases of the invention can be much lower than was hitherto understood in the art.

The invention also embraces treatment as a prophylactic measure.

The term “prophylactically effective amount,” as used herein, pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of a condition, or prior to the worsening of such a condition, with the aim of preserving health by helping to delay, mitigate or avoid that particular condition.

The term “treatment” includes “combination” treatments and therapies, in which two or more treatments or therapies are combined, for example, sequentially or simultaneously. These may be symptomatic or disease modifying treatments.

The particular combination would be at the discretion of the physician.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

In other embodiments the treatment is a “monotherapy”, which is to say that the MT-containing compound is not used in combination (within the meaning discussed above) with another active agent.

For treatment of methaemoglobinaemia, a treatment regimen based on the MT compounds described herein will preferably extend over a sustained period of time appropriate to the disease and symptoms. The particular duration would be at the discretion of the physician.

For example, the duration of treatment may be:
1 to 14, e.g. 1, 2, 3, 4, 5, 6, 7, 8, 9, 10, 11, 12, 13 or 14 days.
1 to 4, e.g. 1, 2, 3 or 4 weeks.

In all cases the treatment duration will generally be subject to advice and review of the physician.

The MT compound of the invention, or pharmaceutical composition comprising it, may be administered to the stomach of a subject/patient orally (or via a nasogastric tube).

Typically, in the practice of the invention the compound will be administered as a composition comprising the compound, and a pharmaceutically acceptable carrier or diluent.

In some embodiments, the composition is a pharmaceutical composition (e.g., formulation, preparation, medicament) comprising a compound as described herein, and a pharmaceutically acceptable carrier, diluent, or excipient.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

One aspect of the present invention utilises a dosage unit (e.g., a pharmaceutical tablet or capsule) comprising an MT compound as described herein (e.g., obtained by, or obtainable by, a method as described herein; having a purity as described herein; etc.), and a pharmaceutically acceptable carrier, diluent, or excipient.

The “MT compound”, although it may be present in relatively low amount, is the active agent of the dosage unit, which is to say is intended to have the therapeutic or prophylactic effect in respect of methaemoglobinaemia. Rather, the other ingredients in the dosage unit will be therapeutically inactive e.g. carriers, diluents, or excipients.

Thus, preferably, there will be no other active ingredient in the dosage unit, no other agent intended to have a therapeutic or prophylactic effect in respect of a disorder for which the dosage unit is intended to be used, other than in relation to the combination treatments described herein.

In some embodiments, the dosage unit is a tablet.

In some embodiments, the dosage unit is a capsule.

In some embodiments, said capsules are gelatine capsules.

In some embodiments, said capsules are HPMC (hydroxypropylmethylcellulose) capsules.

The appropriate quantity of MT in the composition will depend on how often it is taken by the subject per day, or how many units are taken at one time. Therefore dosage units may individually contain less than the total daily dose.

An example dosage unit may contain 10, 20, 30, 40, 50, or 60 mg of MT.

Using the weight factors described or explained herein, one skilled in the art can select appropriate amounts of an MT-containing compound to use in oral formulations.

As explained above, the MT weight factor for LMTM is 1.67. Since it is convenient to use unitary or simple fractional amounts of active ingredients, non-limiting example LMTM dosage units may include 17 mg to provide 10 mg of LMT or MT etc.

In one embodiment there is provided a dosage unit pharmaceutical composition which comprises about 17, 34, 51 mg etc. of LMTM.

The unit dosage compositions described herein (LMTX compound plus optionally other ingredients) may be provided in a labelled packet along with instructions for their use.

In one embodiment, the pack is a bottle, such as are well known in the pharmaceutical art. A typical bottle may be made from pharmacopoeial grade HDPE (High-Density Polyethylene) with a childproof, HDPE pushlock closure and contain silica gel desiccant, which is present in sachets or canisters. The bottle itself may comprise a label, and be packaged in a cardboard container with instructions for us and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding treatment of methaemoglobinaemia.

### Methods of Treatment

Another aspect of the present invention, as explained above, pertains to a method of treatment of methaemoglobinaemia comprising administering to a patient in need of treatment a prophylactically or therapeutically effective amount of a compound as described herein, preferably in the form of a pharmaceutical composition.

### Use in Methods of Therapy

Another aspect of the present invention pertains to a compound or composition as described herein, for use in a method of treatment of methaemoglobinaemia of the human or animal body by therapy.

### Use in the Manufacture of Medicaments

Another aspect of the present invention pertains to use of an MT compound or composition as described herein, in the manufacture of a medicament for use in treatment of methaemoglobinaemia.

In some embodiments, the medicament is a composition e.g. a dose composition as described herein.

### Mixtures of Oxidised and Reduced MT Compounds

The LMT-containing compounds utilised in the present invention may include oxidised (MT⁺) compounds as ‘impurities’ during synthesis, and may also oxidize (e.g., autoxidize) after synthesis to give the corresponding oxidized forms. Thus, it is likely, if not inevitable, that compositions comprising the compounds of the present invention will contain, as an impurity, at least some of the corresponding oxidized compound. For example an “LMT” salt may include up to 15% e.g. 10 to 15% of MT⁺ salt.

When using mixed MT compounds, the MT dose can be readily calculated using the molecular weight factors of the compounds present.

### Salts and Solvates

Although the MT-containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

Naturally, solvates or hydrates of salts of the compounds are also encompassed by the present invention.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1: Changes in oxygen saturation levels in patients receiving LMTM, comparing pre-dose and after four-hours (post-dose) following administration of single doses of LMT at 4 mg and high doses (75/100/125 mg indicated as the mean, 100 mg). Data represent mean (%)±S.E.

FIG. 2: Changes in oxygen saturation levels in patients receiving LMTM over a 6-week period. Pooled data for all doses represent mean (%)±S.E.

FIG. 3: Changes in methaemoglobin levels in patients receiving LMTM, comparing pre-dose and after four-hours (post-dose) following administration of single doses of LMT at 4 mg and high doses (75/100/125 mg indicated as the mean, 100 mg). Data represent mean (%)±S.E.

FIG. 4: Alterations in methaemoglobin levels in patients receiving low (8 mg/day) and high doses (150 mg, 200 mg & 250 mg) of HMTM over a 6-week period. Data represent mean (%)±S.E.

FIG. 5: The implied effect of LMTM treatment on the oxygen-haemoglobin dissociation curve as described in the text. Data for patients with mild hypoxaemia come from subjects treated with LMTM. Data for patients with severe hypoxaemia come from results reported by Hamidi-Alamdari, D., et al. “Methylene blue for treatment of hospitalized COVID-19 patients, randomized, controlled, open-label clinical trial, phase 2.” Revista de Investigacion Clinica. 2021; 73:190-198. in which a preparation of reduced MTC was used to deliver LMT orally.

FIG. 6: Computational chemistry modelling of the high affinity LMT/MT⁺-haem interaction. (A&B) Heme in the nonplanar unbound T-state showing protrusion of the iron atom above the plane of the porphyrin ring. (C&D) Heme in the flat oxygen-bound R-state. (E&F) LMT binding results in heme adopting the R-state.

### EXAMPLES

### Example 1—Methylthioninium Chloride (MTC) and LMTX

MTC (methylthioninium chloride, methylene blue) has been available as a drug since 1876. It is on the world health organisation's list of essential medicines, which is a list of the safest and most effective medicines in a health system.

MTC has been applied previously in many areas of clinical medicine including treatment of methemoglobinemia, malaria, nephrolithiasis, bipolar disorder, ifosfamide encephalopathy and most recently in Alzheimer disease (AD) (see “Wischik, C. M., et al. “Tau aggregation inhibitor therapy: an exploratory phase 2 study in mild or moderate Alzheimer's disease” Journal of Alzheimer's Disease 2015; 44:705-720.” and “Nedu, M. E., et al. Comparative study regarding the properties of methylene blue and proflavine and their optimal concentrations for in vitro and in vivo applications.” Diagnostics 2020; 10:223.”).

The MT moiety can exist in the oxidised MT⁺ form and in the reduced LMT form (see Harrington et al., 2015; supra).

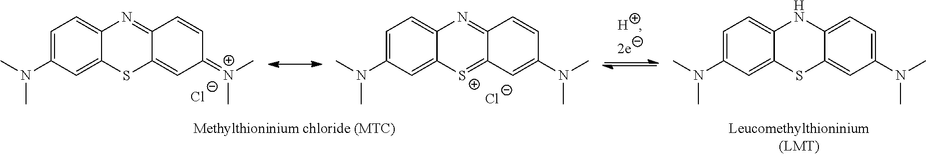

MTC is the chloride salt of the oxidised MT⁺ form. It needs to be converted to the reduced leuco-MT (LMT; international non-proprietary name: hydromethylthionine) form by a thiazine dye reductase activity in the gut to permit absorption and distribution to deep compartments including red cells and brain (see Baddeley et al., 2015; supra). Likewise, in isolated red cell preparations, MT⁺ needs to be converted to LMT to permit uptake both into red cells (see “May, J. M., et al., “Reduction and uptake of methylene blue by human erythrocytes.” American Journal of Physiology—Cell Physiology 2004; 286:C1390-C1398.”) and into pulmonary endothelial cells (Merker et al., 1997; supra).

Because MTC is actually a prodrug for LMT, the predominant form in the body, TauRx developed a stabilised reduced form of MT as LMTM (leuco-methylthioninium bis(hydromethanesulphonate); hydromethylthionine mesylate) in order to permit direct administration of the LMT form.

Synthesis of LMTX and LMTM compounds can be performed according to the methods described in the art (see e.g. WO2007/110627, and WO2012/107706)

### Example 2—Summary of Investigation

Objective: To determine the effects of oral LMTM on SPO₂ and metHb levels in patients with mild hypoxaemia not due to COVID-19 and explore the structural interaction between the LMT moiety and the haem group.

Methods: Eighteen AD trial participants randomised to unit 4/75/100/125 mg doses of LMTM had SPO₂ levels below 94% at baseline. Patients were routinely monitored by pulse oximetry after 4 hrs, and after 2 and 6 weeks of twice daily dosing. Computational chemistry was used to understand LMT-heme binding.

### Example 3—Trial Methods and Results

### Methods

### Patients

The study design and results from two Phase 3, double-blind, controlled, randomised, studies of LMTM as a potential treatment for AD were described elsewhere (see “Gauthier, S., et al. “Efficacy and safety of tau-aggregation inhibitor therapy in patients with mild or moderate Alzheimer's disease: a randomised, controlled, double-blind, parallel-arm, phase 3 trial.” Lancet 2016; 388:2873-2884.” and “Wilcock, G K, et al. “Potential of low dose leuco-methylthioninium bis(hydromethanesulphonate) (LMTM) monotherapy for treatment of mild Alzheimer's disease: cohort analysis as modified primary outcome in a Phase Ill clinical trial.” Journal of Alzheimer's Disease 2017; 61:435-457.). In summary, 890 mild/moderate AD patients were randomly assigned (3:3:4) to 150 mg/day, 250 mg/day or 8 mg/day (intended as a control) for 15-months (clinicaltrials.gov NCT01689246). Similarly, 800 patients with mild AD were randomly assigned to 200 mg/day or 8 mg/day LMTM for 18-months (clinicaltrials.gov NCT01689233). In both trials, MetHb and SpO₂ levels were measured by pulse oximetery (Massimo Corporation rad 57) at screening, baseline (within 1 hour prior to dosing), post-dose during the 4-hour observation, and subsequent clinic visits at 2 and 6 weeks.

### Estimation of Effect of LMT Treatment on Oxygen-Haemoglobin Dissociation Curve

We have used a standard clinical SpO₂ to PaO₂ conversion table (e.g. https://www.acphospitalist.org/archives/2013/11/acph-201311-coding_t1.pdf) to compare the oxygen-haemoglobin dissociation curves in patients receiving LMTM or those receiving a reduced form of MTC (see “Hamidi-Alamdari, D., et al. “Methylene blue for treatment of hospitalized COVID-19 patients, randomized, controlled, open-label clinical trial, phase 2.” Revista de Investigacion Clinica. 2021; 73:190-198.”) with normal. For patients treated with LMTM, implied PaO₂ values at baseline and corresponding SpO₂ values were plotted. After treatment with LMTM, the SPO₂ values were plotted at the same PaO₂ values to deduce the implied shift in the dissociation curve. In order to compare these treatment effects with those reported by Hamidi-Alamdari et al. (2021) supra, SpO₂ values observed in patients receiving standard of care were used to calculate the implied untreated PaO₂ values. The SpO₂ values observed after 5 days of treatment with the reduced MTC preparation were plotted at the same PaO₂ values to infer the implied shift in oxygen-haemoglobin dissociation.

### Statistics

Statistical analyses were performed using R-version 3.5.1, employing paired samples t-tests to compare mean SpO₂ and metHb levels.

### Computational Modelling

Quantum chemistry calculations were performed using the self-consistent field (SCF) method within the MOE software suite, Chemical Computing Group, University College London. SCF calculations used the Austin Model-1 basis set, and geometry optimisation were initially performed using the AMBER 10 force field using Extended Hückel Theory. The crystal structure of human haemoglobin in the oxy form (2DN1) was used to align the LMT-heme complex (see “Park, S.-Y., et al. “1.25 Å resolution crystal structures of human haemoglobin in the oxy, deoxy and carbonmonoxy forms.” Journal of Molecular Biology 2006; 360:690-701.”). The crystal structure of human haemoglobin in the deoxy form (PDB ID 2DN2; Park et al., 2006) and the crystal structure of human haemoglobin in the carbonmonoxy form (PDB ID 3DN2; (Park et al. 2006) were aligned using Pymol.

### Results

### Population Characteristics at Baseline

Data were available for 18 subjects from both AD studies who had oxygen saturation<94% at baseline (lower limit of normal range is 95%). Their demographic characteristics are summarised in Table 1. Mean age was 76.1 years, ranging from 70 to 80 years, with more females (61%) than males (39%).

Using medical history available from patient Case Report Forms, hypoxemia in these subjects was found to be associated with a variety of underlying respiratory or other conditions of varying degrees of severity which could plausibly have contributed to chronic hypoxaemia (summarised in Table 2). These include sleep apnoea, insomnia, asbestosis, oedema, asthma, bronchitis, allergies, angioedema, pneumonia, acute myocardial infarction/hypertension, coronary artery disease with angioplasty and stent insertion, transient ischaemic attacks (TIA), hypothyroidism, diabetes, syncope, tachycardia and sepsis. No predisposing clinical history factors could be identified in three of the subjects.

Prospective Clinical Study of Peripheral Oxygen Saturation and Methaemoglobinaemia in AD Patients Treated with LMTM

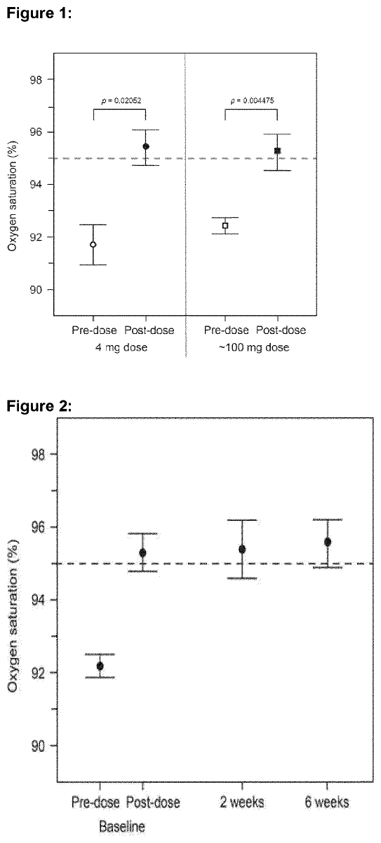

SpO₂ levels were compared pre-dose and after four hours in the clinic following administration of a single dose of LMTM at 4 mg or doses of 75, 100 or 125 mg (summarised as the mean, 100 mg, for the high doses; FIG. 1). As can be seen in FIG. 1, LMTM at a dose of 4 mg substantially increased mean blood oxygen saturation levels. Mean oxygen saturation in the group with baseline SpO₂ levels below 94% was 91.71%. Four hours after receiving a 4 mg dose of LMTM, the mean SpO₂ level was increased to 95.43% (+3.72%, p=0.0205; Table 3, FIG. 1). Likewise, following administration of high dose LMTM, oxygen saturation levels increased from 92.45% at baseline to 95.27% after four hours (+2.82%, p=0.0045; Table 3, FIG. 1). Therefore, LMTM is able to increase blood oxygen saturation within 4 hours across a broad range of doses with no discernible dose-dependent differences. As can be seen from FIG. 2 and Table 4, the effect is stable over 6 weeks at 8 mg/day and 150-250 mg/day and the differences with respect to baseline were statistically significant at week 2 (+3.17%±se, p=0.0034) and week 6 (+3.23%±se, p=0.0005). Therefore, LMTM produces a rapid improvement in oxygen saturation at single doses in the range 4-125 mg, and this is sustained over 6 weeks at doses in the range 8-250 mg/day.

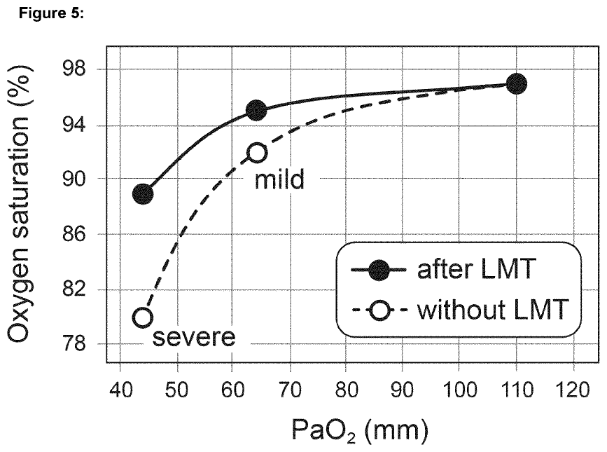

The implied effect of treatment on the oxygen-haemoglobin dissociation curve is shown in FIG. 5. We have used a standard dissociation curve to estimate the corresponding PaO₂ value at baseline. It can be seen that the effect of LMTM treatment in raising the SpO₂ value corresponds to an implied left-shift in the oxygen-haemoglobin dissociation curve. The cases in which LMTM treatment were used had relatively mild hypoxaemia (FIG. 5, ‘mild’). For more severe cases (FIG. 5, ‘severe’) we have used the same approach to infer the implied effect on oxygen-haemoglobin dissociation in patients receiving the reduced MTC formulation reported by Hamidi-Alamdari et al. (2021) (supra) using the untreated population as a basis for comparison.

As can be seen, the implied effect of treatment with reduced MTC is similar.

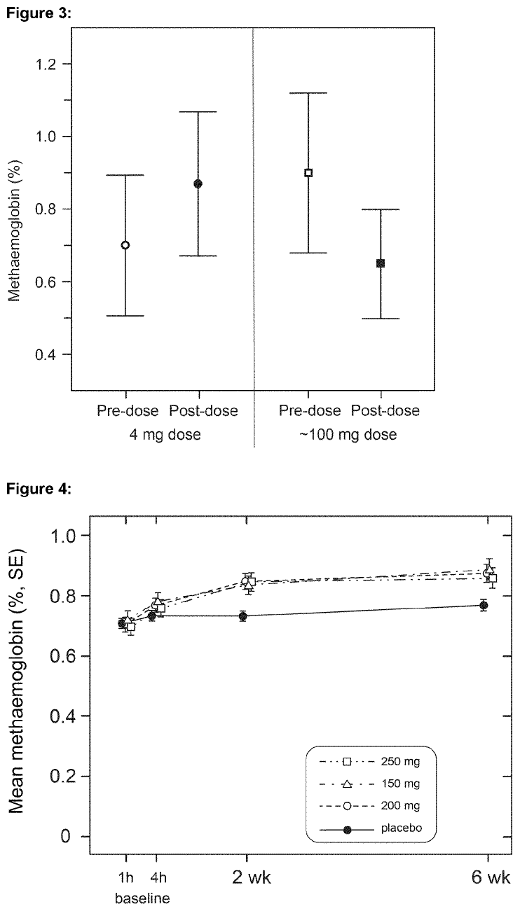

We next investigated how the effect on hypoxaemia relates to metHb levels measured simultaneously in the same patients. For both treatment groups, there was no consistent difference in metHb levels between baseline and 4-hours post-dose (FIG. 3, Table 3). Therefore, the acute effect of LMTM on oxygen saturation is independent of any consistent corresponding effect on methaemoglobin at either low or high doses. Over 6 weeks, LMTM at the higher doses (150, 200 and 250 mg/day) systematically increased the metHb levels but not at the 8 mg/day dose (FIG. 4, Table 5A), although none of the changes in metHb reached statistical significance in this small number of subjects. When the effects on metHb levels were considered in the entire population, the small increases became statistically significant, including at the 8 mg/day dose (Table 5B). However it will be appreciated that, in terms of therapeutic efficacy, these small changes (around 0.7% to 1%) are not consequential in relation to addressing metHb at levels seen in symptomatic patients e.g. >20%, or >30% metHb (see Table 7).

### Computational Chemistry Model of LMT-Heme Effect

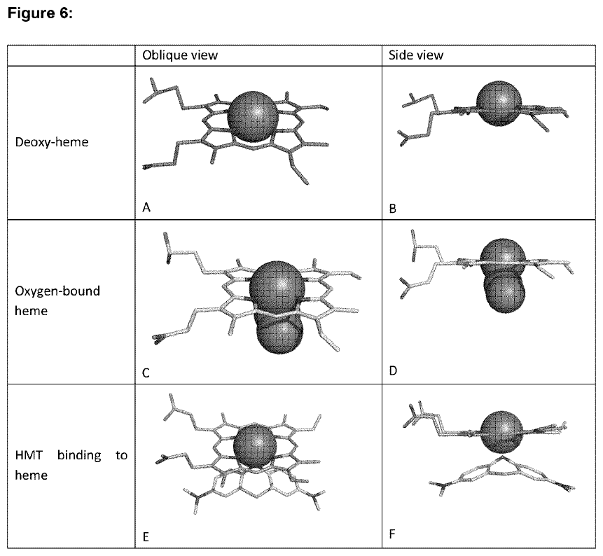

We have used computational modelling to study the minimum-energy LMT-heme. The modelling suggests that LMT is able to bind with high affinity to the heme iron of haemoglobin via the LMT nitrogen in an octahedral geometry and within 2.1 Å of the iron atom (FIG. 6). LMT interacts strongly with the d_{z}_{²} and dₓ_{²}_{-y}_{²} orbitals of the Fe²⁺ electrons, which are oriented at the axial ends and equatorial corners, respectively, of the octahedral complex. We hypothesize that the formation of this LMT-heme complex acts in a manner analogous to binding of oxygen (FIGS. 6C&D) in helping to overcome the initial barrier for oxygen binding by facilitating a shift from the T-state (FIGS. 6A&B) to the R-state (FIGS. 6E&F). The resulting co-operativity would permit higher relative oxygen saturation to occur at lower PaO₂ levels, consistent with the implied left-shift in the oxygen-haemoglobin dissociation curve suggested by our analyses.

### Example 4—Discussion of Example 3

We report above the preliminary results of an exploratory analysis of the effects of LMTM treatment on patients who were chronically mildly hypoxaemic at baseline.

We investigated 18 subjects who came into either of two phase 3 trials in mild to moderate AD and who had oxygen saturation levels below 94% at baseline, due to a range of incidental chronic cardiorespiratory conditions. We show that single doses of LMTM in the range 4-125 mg were able to increase oxygen saturation levels significantly within 4 hours, and that the effect persists over 6 weeks of treatment with the same doses given twice daily. This implies that LMTM is able to bind to haemoglobin in such a way as to enhance oxygen saturation by about 3% in patients with chronic hypoxaemia.

The paradoxical interaction of the MT moiety in relation to methaemoglobinaemia has already been discussed above. While the LMT species acts to reduce Fe³⁺ to Fe²⁺ in methaemoglobinaemia (see “May, J. M., et al. “Reduction and uptake of methylene blue by human erythrocytes.” American Journal of Physiology—Cell Physiology 2004; 286:1390-1398.” and “Schirmer, R. H., et al. “Lest we forget you—methylene blue . . . ” Neurobiology of Aging 2011; 32:2325.e7-2325.e16.”), both the MT and LMT species can themselves cause methaemoglobinaemia.

We have utilised computation chemistry to understand the anti-hypoxaemia effect of LMT. As both the structure of heme and of LMT are known, it is possible to compute the minimum-energy binding interaction between the two. We show that LMT is able to bind with high affinity within 2.10 Å of the iron atom of haemoglobin by donating a pair of electrons from the central nitrogen of LMT to the d_{z}_{²} and dₓ_{²}_{-y}_{²} orbitals of the Fe²⁺ electrons. From crystal field theory, this type of interaction has an estimated field factor of 1.2-1.5 (see “Jorgensen, C. K. “Absorption spectra and chemical bonding in complexes.” Pergamon Press, Oxford, London, New York, Paris 1962. 352pp.” and “Jorgensen, C. K. “Oxidation numbers and oxidation states. Springer Berlin Heidelberg; 1969.”), implying that LMT is able to act as a strong displaceable field ligand. We hypothesize that the formation of this complex facilitates a shift from the T-state in which Fe²⁺ has an ionic radius of 2.06 Å, which is too large to fit into the cavity in the centre of the porphyrin ring (see “Perutz, M. F. “Proteins and nucleic acids-structure and function.” Amsterdam and New York: Elsevier Publishing Co.; 1962.”), to the R-state in which Fe²⁺ has an ionic radius of 1.96 Å enabling it to fit within the four nitrogen atoms with which it coordinates. Oxygen is able to bind with higher affinity to R-state heme than T-state heme (see “Lima, F. A., et al.” “Probing the electronic and geometric structure of ferric and ferrous myoglobins in physiological solutions by Fe K-edge absorption spectroscopy.” Physical Chemistry Chemical Physics 2014; 16:1617-1631.”), thereby overcoming the initial energy barrier, and subsequent binding of oxygen is further facilitated by cooperativity (see “Bohr, C., et al. “Ueber einen in biologischer Beziehung wichtigen Einfluss, den die Kohlensäurespannung des Blutes auf dessen Sauerstoffbindung übt.” Skandinavisches Archiv für Physiologie 1904; 16:402-412.”. This is consistent with a left-shift of the oxygen-haemoglobin dissociation curve (28).

However, the LMT binding is non-optimal compared with oxygen. Whereas the binding distance between the LMT nitrogen and heme iron is 2.10 Å, the corresponding binding distance for oxygen is it is 1.98 Å. This implies that oxygen would be able to displace LMT when it is available at high pH or low pCO₂, thereby permitting normal oxygen dissociation to occur with release of bound oxygen to peripheral tissues. This is consistent with the reduction in respiratory rate observed in severely ill patients treated with the reduced MTC preparation of LMT (Hamidi-Alamdari, D. et al., 2021, supra). Respiratory rate is driven by central and peripheral chemoreceptors sensitive to hypoxia and increased CO₂ levels which signal tissue hypoxia (see “Davies, A. and Moores, C. “The Respiratory System: Basic science and clinical conditions.” 2nd Edition. Churchill Livingstone; 2010.”.

The formation of the heme-LMT complex that we describe provides a structural explanation for three different possible effects of the MT moiety on haemoglobin: the reduction Fe³⁺ to Fe²⁺ in methaemoglobinaemia (13), the conversion of Fe²⁺ to Fe³⁺ at high concentrations of MT (see “Bodansky, O. “Methemoglobinemia and methemoglobin-producing compounds.” Pharmacological Reviews 1951; 3:144-196.”), and the effect on oxygen saturation we describe here. In the first two, the formation of the heme-LMT coordinate permits either donation of an electron or removal of an electron, depending on the availability of adequate levels of NADPH needed to regenerate LMT. This is required to convert the MT⁺ produced from LMT during reduction of Fe³⁺ back to LMT. At high concentrations of the MT moiety (or in the presence of G6PD deficiency), the level of MT⁺ exceeds the available reducing capacity of the cell. MT⁺, which is able to form the same co-ordinate with heme via orientation of the nitrogen atom, oxidises Fe²⁺ to Fe³⁺ and forms LMT. In the case of hypoxaemia occurring in the context of normal red cell physiology, we show that low doses of LMTM are able to improve oxygen saturation with minimal corresponding effect on levels of metHb. At higher doses of LMTM, there is still enhancement of oxygen saturation, but in addition, Fe²⁺ is oxidised to Fe³⁺.

It is proposed that the use of LMTM (and related compounds) at oral doses of more than the control dose (4 mg) but less than the higher doses (75 mg) tested herein should provide for net reduction of Fe³⁺ to Fe²⁺ in methaemoglobinaemia (hereditary and acquired forms). Such an effect would be consistent with the structural model and data described herein, and provide a valuable addition to the treatment options currently available.

TABLE 1
Clinical characteristics of patients at baseline.

| | Hydromethylthionine |
|---|---|
| Characteristic | Total (n = 18) |
| Age (y) | |
| Mean (SD) | 76.1 (7.8) |
| Median (range) | 77.5 (70-80) |
| Sex n (%) | |
| Male | 7 (39%) |
| Female | 11 (61%) |
| Race n (%) | |
| Black or African American | 2 (11%) |
| White | 16 (89%) |

TABLE 2
Presenting clinical history of clinical trial
subjects presenting with low oxygen saturation.

| | Clinical respiratory/ | Aggravating clinical |
|---|---|---|
| Subject | ventilation type | conditions¹ |
| 1 | Sleep Apnoea | hypothyroid/diabetic |
| 2 | Insomnia | Often a sign of obstructive sleep |
| | | apnoea or other mild hypoxia |
| | | conditions such as paroxysmal |
| | | nocturnal dyspnea |
| 3 | | Hypertension/left bundle branch |
| | | block (LBBB); |
| | | Left ventricular hypertrophy (LVH) |
| 4 | Asbestosis | |
| 5 | Oedema | Often a sign of right heart failure |
| | | or congestive cardiac failure/may |
| | | also be simple sedentary dependent |
| | | oedema |
| 6 | | Hypertension/LBBB/LVH |
| 7 | | Acute myocardial infarction/ |
| | | Hypertension |
| 8 | | Hypertension |
| 9 | | Hypertension/Coronary artery disease |
| | | with angioplasty and stent insertion |
| 10² | | |
| 11² | | |
| 12 | Asthma (childhood) | |
| 13 | Sleep apnoea with | |
| | uvulectomy | |
| 14 | Bronchitis/Seasonal | |
| | allergies/Anaemia | |
| 15 | | Acute myocardial infarction with |
| | | stent insertion |
| 16² | | |
| 17 | | Transient ischaemic attack/ |
| | | Hypertension |
| 18 | Asthma (childhood)/ | Hypertension/hypothyroid/syncope/ |
| | Intermittent angioedema/ | tachycardia/sepsis |
| | Occasional insomnia/ | |
| | Pneumonia | |

¹Aggravating clinical conditions that may also cause hypoxia if sufficiently severe or chronic.
²Three patients had no predisposing clinical history factors listed.

TABLE 3
Change from pre-dose to post-dose oxygen saturation
and methaemoglobin levels following administration
of LMT at a dose of 4 mg or high doses (75/100/150
mg indicated as 100 mg). Data represent mean (%) ± S.E.

| | 4 mg | Post | 100 mg | Post |
|---|---|---|---|---|
| | Pre-dose | dose | Pre-dose | dose |
| Oxygen saturation, | 91.71 | 95.43 | 92.45 | 95.27 |
| mean % (SE) | (0.75) | (0.65) | (0.28) | (0.74) |
| Methaemoglobin, | 0.70 | 0.87 | 0.90 | 0.65 |
| mean % (SE) | (0.195) | (0.198) | (0.220) | (0.154) |

TABLE 4
Change in oxygen saturation levels in pooled data
for patients receiving LMTM at Baseline and over
a 6-week period. Data represent mean (%) ± S.E.

| | Oxygen | |
|---|---|---|
| Visit | saturation | p-value |
| Baseline (1 hr), | 92.167 | |
| mean % (SE) | (0.336) | |
| Baseline (4 hr), | 95.333 | 0.000139 |
| mean % (SE) | (0.505) | |
| Week 2, mean % | 95.400 | 0.0034 |
| (SE) | (0.798) | |
| Week 6, mean % | 95.571 | 0.0005 |
| (SE) | (0.618) | |

Table 5: (A) Change in methaemoglobin levels in the subgroup of patients with baseline oxygen saturation levels below 94% receiving LMTM at low (8 mg/day) or high dosages (150/200/250 mg/day) over a 6-week period. (B) Change in methaemoglobin levels in all patients with available data receiving LMTM at low (8 mg/day) or high dosages (150/200/250 mg/day) over a 6-week period. Data represent mean (%)±S.E.

TABLE 5
A (patients with SpO₂ < 94% at baseline):

LMT dose

| Visit | 4 mg | 150 mg | 200 mg | 250 mg |
|---|---|---|---|---|
| Baseline, mean % | 0.709 | 0.721 | 0.702 | 0.696 |
| (SE) | (0.017) | (0.029) | (0.025) | (0.028) |
| 4 hr, mean % | 0.733 | 0.781 | 0.760 | 0.758 |
| (SE) | (0.018) | (0.030) | (0.023) | (0.028) |
| Week 2, mean % | 0.732 | 0.835 | 0.849 | 0.845 |
| (SE) | (0.018) | (0.032) | (0.027) | (0.033) |
| Week 6, mean % | 0.769 | 0.888 | 0.873 | 0.858 |
| (SE) | (0.019) | (0.034) | (0.030) | (0.033) |

TABLE 5
B (all patients):

| | 8 mg | 150 mg | 200 mg | 250 mg |
|---|---|---|---|---|
| Baseline, mean | 0.709 ± 0.017 | 0.721 ± 0.029 | 0.702 ± 0.025 | 0.696 ± 0.028 |
| % ± SE (N) | (754) | (268) | (398) | (265) |
| 4 hr, mean | 0.732 ± 0.017 | 0.7811 ± 0.029 | 0.760 ± 0.023 | 0.758 ± 0.028 |
| % ± SE (N) | (754) | (267) | (398) | (263) |
| p-value | 0.09732 | 0.02212 | 0.01967 | 0.005868 |
| Week 2, mean | 0.733 ± 0.018 | 0.831 ± 0.032 | 0.849 ± 0.027 | 0.837 ± 0.034 |
| % ± SE (N) | (721) | (248) | (355) | (239) |
| p-value | 0.1326 | <0.0001 | <0.0001 | <0.0001 |
| Week 6, mean | 0.770 ± 0.019 | 0.889 ± 0.034 | 0.873 ± 0.030 | 0.851 ± 0.033 |
| % ± SE (N) | (721) | (248) | (355) | (239) |
| p-value | 0.0006916 | <0.0001 | <0.0001 | <0.0001 |

TABLE 6
(see Iolascon 2021, supra):

| | | | | | | | | Hb |
|---|---|---|---|---|---|---|---|---|
| | | | | Other | | MetHb | CYB5R | electrophoresis/ |
| Disease | Transmission | Cyanosis | Anemia | symptoms | Gene(s) | level % | activity | HPLC |
| Drug exposure | Acquired | Yes | No | — | — | Variable | Normal | Normal |
| Methemoglobinemia, | Autosomal | Yes | No | — | CYB5R3 | 20-30 | Decreased | Normal |
| type I | recessive | since | | | | | | |
| | | birth | | | | | | |
| Methemoglobinemia, | Autosomal | Yes | No | Neurological | CYB5R3 | 8-40 | Decreased | Normal |
| type II | recessive | since | | involvement | | | | |
| | | birth | | | | | | |
| Methemoglobinemia, | Autosomal | Yes | No | 46, XY DSD | CYB5A | 12-19 | Normal | Normal |
| type IV | recessive | | | Ambiguous | | | | |
| | | | | genitalia | | | | |
| HbM disease | Autosomal | Yes | Yes | — | HBA1, | 12-25 | Normal | Abnormal |
| | dominant | since | | | HBA2, | | | |
| | | birth or | | | HBB, | | | |
| | | after | | | HBG1, | | | |
| | | HbF/A | | | HBG2 | | | |
| | | switching | | | | | | |
| Unstable Hb | Autosomal | Yes | Yes | — | HBA1, | Variable | Normal | N or Abnormal |
| | dominant | | | | HBA2, | (Stressor | | |
| | | | | | HBB, | induced) | | |
| | | | | | HBG1, | | | |
| | | | | | HBG2 | | | |

Abbreviation: DSD, disorder of sexual differentiation.

TABLE 7
(see Iolascon 2021, supra):

| MetHb | | | |
|---|---|---|---|
| level | Signs | Symptoms | Causes |
| <10% | Low pulse oximeter | Asymptomatic | Acquired |
| | readings, alteration of | | |
| | the skin color (pale, | | |
| | gray, blue) | | |
| 10%-30% | Cyanosis | Asymptomatic/ | Enzymopenic |
| | Dark brown blood | confusion | methaemoglobinaemia, |
| | | | HbM (M group |
| | | | variants of Hb), |
| | | | acquired |
| 30%-50% | Dyspnea, dizziness, | Confusion, chest pain, | Acquired ± hereditary |
| | syncope | palpitations, headache, | |
| | | fatigue | |
| 50%-70% | Tachypnea, metabolic | Confusion, chest pain, | Acquired ± hereditary |
| | acidosis, dysrhythmias, | palpitations, headache, | |
| | seizure, delirium, coma | fatigue | |
| >70% | Severe hypoxemia, | — | Acquired ± hereditary |
| | death | | |

1. A method of treating methaemoglobinaemia in a subject,
which method comprises orally administering to said subject a methylthioninium (MT)-containing compound,
wherein said administration provides a total daily oral dose of 4 mg to 60 mg of MT to the subject per day, optionally split into 2 or more doses,
wherein the MT-containing compound is an LMTX compound of the following formula: 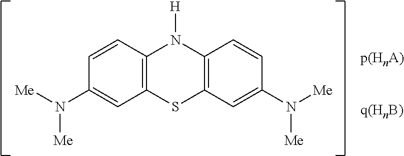
wherein each of HₙA and HₙB (where present) are protic acids which may be the same or different,
and wherein p=1 or 2; q=0 or 1; n=1 or 2; (p+q)×n=2,
or a hydrate or solvate thereof.

2. A method as claimed in claim 1 wherein the total daily dose is about 4, 8, 12, 16, 20, 20.5, 21, 21.5, 22, 22.5, 23, 23.5, 24 mg to around any of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 mg.

3. A method as claimed in claim 1 or claim 2 wherein the total daily dose is between 8 or 10 or 20 or 20.5 or 21 and 50 or 55 or 60 mg.

4. A method as claimed in claim 1 wherein the total daily dose is about 8, 12, 16, 20, 20.5, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 mg.

5. A method as claimed in claim 1 wherein the total daily dosage is 10 to 50 mg.

6. A method as claimed in claim 1 wherein the total daily dose is 20.5 to 50 mg.

7. A method as claimed in claim 1 wherein the total daily dosage is 40 to 50 mg.

8. A method as claimed in claim 1 wherein the total daily dosage is about 4, 8, 12, or 16 mg.

9. A method as claimed in any one of claims 1 to 8 wherein the total daily dose of the compound is administered as a split dose twice a day or three times a day.

10. A method as claimed in any one of claims 1 to 9 wherein the subject has presented with a sign or symptom shown in Table 7, but wherein the subject is optionally
(1) not hypoxic or suffering from hypoxaemia;
(2) has a blood oxygen saturation level (SpO2) of more than 95% on room air.

11. A method as claimed in claim 10 comprising the step of selecting the subject according to said sign or symptom and/or genetic testing for a gene shown in Table 6.

12. A method as claimed in any one of claims 1 to 11 wherein the methaemoglobinaemia is acquired, and optionally the subject has a methaemoglobin level of >30%, >50% or >70%.

13. A method as claimed in any one of claims 1 to 11 wherein the methaemoglobinaemia hereditary, and optionally the subject has a methaemoglobin level of >30%, >50% or >70%.

14. A method as claimed in any one of claims 1 to 13 wherein the MT-containing compound has the following formula, where HA and HB are different mono-protic acids:

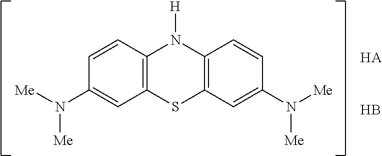

15. A method as claimed in any one of claims 1 to 13 wherein the MT-containing compound has the following formula: 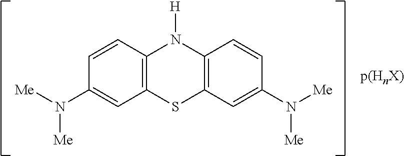
wherein each of HₙX is a protic acid.

16. A method as claimed in any one of claims 1 to 13 wherein the MT-containing compound has the following formula and H₂A is a di-protic acid:

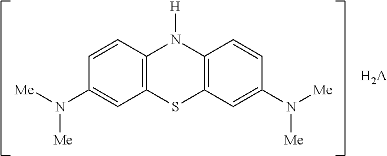

17. A method as claimed in claim 15 wherein the MT-containing compound has the following formula and is a bis-monoprotic acid:

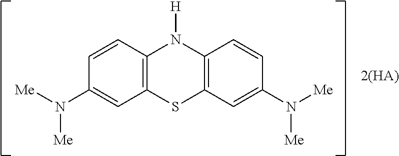

18. A method as claimed in any one of claims 1 to 17 wherein the or each protic acid is an inorganic acid.

19. A method as claimed in claim 18 wherein each protic acid is a hydrohalide acid.

20. A method as claimed in claim 18 wherein the or each protic acid is selected from HCl; HBr; HNO₃; H₂SO₄.

21. A method as claimed in any one of claims 1 to 17 wherein the or each protic acid is an organic acid.

22. A method as claimed in claim 21 wherein the or each protic acid is selected from H₂CO₃; CH₃COOH; methanesulfonic acid, 1,2-ethanedisulfonic acid, ethansulfonic acid, naphthalenedisulfonic acid, p-toluenesulfonic acid.

23. A method as claimed in any one of claims 1 to 17, or claim 22 wherein the MT-containing compound is LMTM:

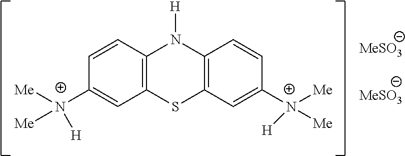

24. A method as claimed in any one of claims 1 to 17 wherein the MT-containing compound is selected from the list consisting of:

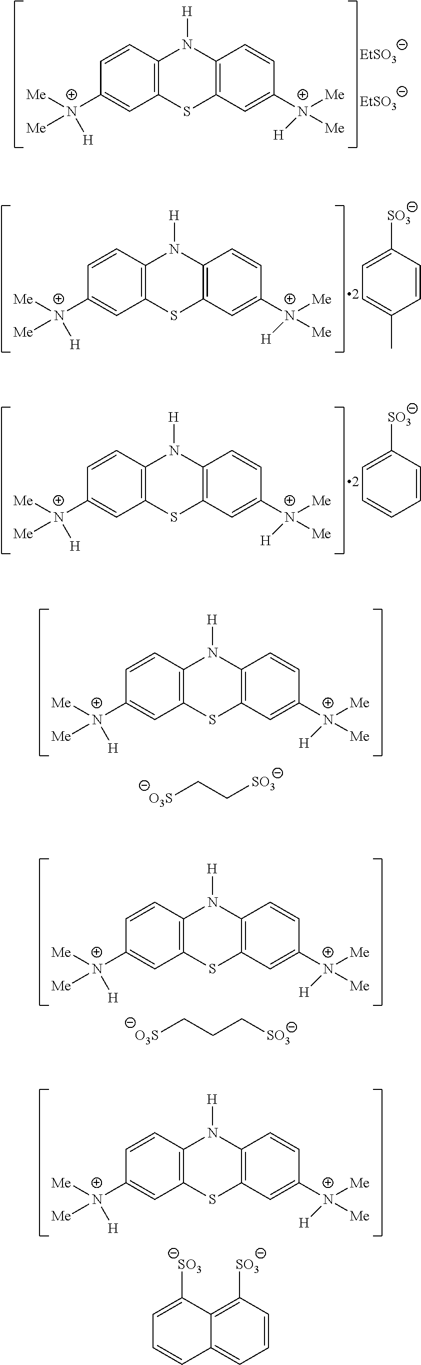

25. An MT-containing compound as defined in any one of claims 1 to 24, for use in a method of treatment as defined in any one of claims 1 to 24.

26. Use of an MT-containing compound as defined in any one of claims 1 to 24, in the manufacture of a medicament for use in a method of treatment as defined in any one of claims 1 to 24.
